(19) United States
(12) Patent Application Publication
(10) Pub. No.: US 2006/0057226 A1
(43) Pub. Date: Mar. 16, 2006

(54) Red ear drops

(76) Inventor: John Moore Johnson, Shannon, MS (US)

Correspondence Address: John Moore Johnson, 144 Dr. 497, Shannon, MS, 38868, US

(21) Appl. No.: 10/943,430

(22) Filed: Sep. 16, 2004

(51) Int. Cl. A61K 33/36

(52) U.S. Cl. 424/672

(57) ABSTRACT

My invention, Red Ear Drops, is a combination of povidone-iodine and lidocaine hydrochloride monohydrate. The prior entity, povidone-iodine, is effective against many pathogens which include viruses, bacteria, fungi, protozoa, and yeast. The latter drug, lidocaine hydrochloride monohydrate is an effective all purpose, local anesthetic agent. The combination drug product can be an effective and safe treatment for many types of ear infections at a reasonable cost to the patient.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### REFERENCE TO SEQUENCE LISTING, A TABLE, OR A COMPUTER PROGRAM LISTING COMPACT DISC APPENDIX

“Not Applicable”

### BACKGROUND OF THE INVENTION

This invention is a medical endeavor to improve the ability to treat otitis media and other irritating ear conditions at a reasonable cost to the patient.

### BRIEF SUMMARY OF THE INVENTION

The two ingredients used in the RED EAR DROPS are used in prescription and over the counter drug products. Most antibacterial ear preparations are expensive. This will give physicians and patients an alternative to the more expensive otic medications. The drops include povidone-iodine and lidocaine. These ingredients will create an effective ear drop against many pathogens and provide pain relief.

### BRIEF DESCRIPTION OF THE SEVERAL VIEWS OF THE DRAWING

The drawing displays the chemical formula of the ingredients used in Red Ear Drops.

### DETAILED DESCRIPTION OF THE INVENTION

The products in RED EAR DROPS and their pharmacological properties are as follows:
A. Povidone-Iodine Solution—water soluble iodine complex that has germicidal activity without irritation to skin or mucous membranes. It has broad microbicidal spectrum against bacteria, fungi, viruses, protozoa, and yeast.
B. Lidocaine—soluble all purpose local anesthetic agent

Amount of ingredients needed to produce 10 gallons of otic solution:
A. Lidocaine HCL Monohydrate—230 grams
B. Povidone-Iodine 10% solution—QS 10 gallons
   Indications for Use—

For treatment of superficial infections and pain of the external auditory ear canal caused by certain bacteria, fungi, viruses, and protozoa, which are susceptible to the action of the above listed ingredients in Red Ear Drops

Precautions—

If improvement is not seen in three days, consult physician for further treatment.

Contraindications—

A. Not for use in children under three years of age unless directed by physician
B. Not for use in the ears in which the tympanic membrane is perforated, except as directed by physician.
C. Not for use in persons who are allergic to any of the ingredients listed in Red Ear Drops. These include iodine products and Lidocaine (including other local anesthetics)
D. Not for use as the only drug product to treat severe inner ear infections. Should be combined with appropriate oral or injectible antibiotic.
   Patient Information—
A. Not for ophthalmic, oral, or injectible use.
B. If contact occurs with clothing or other material, wash immediately.
C. Even if improvement is seen, still complete the 7 day course of therapy.
   Dosage and Administration—

Tilt head so drops will flow into ear canal. Instill 7 drops in ear. Leave head tilted for 20-30 seconds to let solution be absorbed. Place a piece of cotton in ear to keep solution from flowing out of ear when head is straightened. Repeat same steps in other ear if affected. The drops should be instilled in the (ear)s twice daily for seven days.

How Supplied

THE RED EAR DROPS ARE SUPPLIED IN A 10 ML DROPPER BOTTLE.
   THE DROPS SHOULD BE STORED AT ROOM TEMPERATURE
   (59 TO 86 DEGREES FAHRENHEIT)

### BRIEF DESCRIPTION OF THE SEVERAL VIEWS OF THE DRAWING

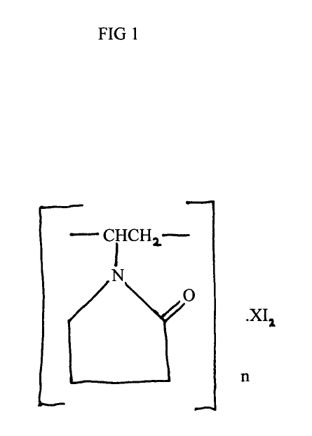

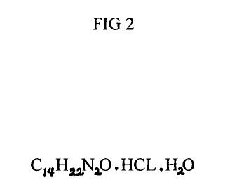

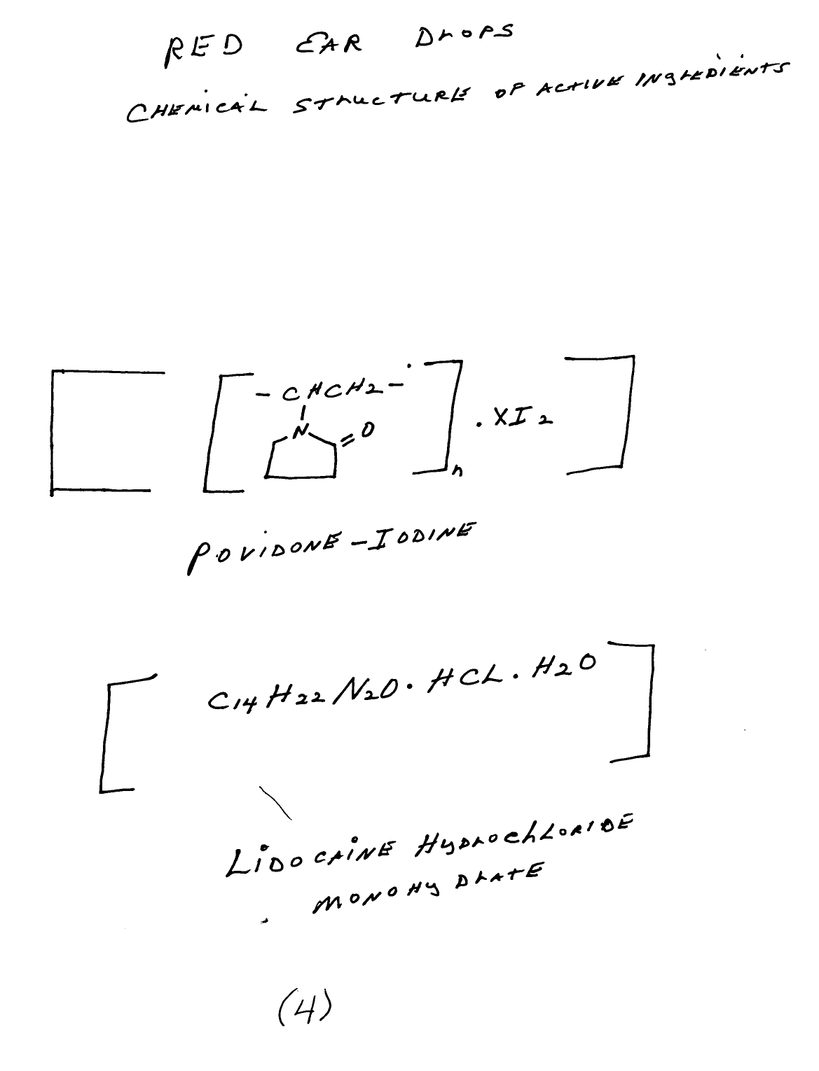

The chemical formula of the two drugs incorporated in Red Ear Drops are depicted on the two following pages: FIG. 1 represents the chemical formula for povidone-iodine. FIG. 2 represents the chemical formula for lidocaine hydrochloride monohydrate.

1. What I claim as my invention is Red Ear Drops, which consists of the use of Povidone-Iodine Solution and Lidocaine in an effective drug product and in a combination that has not previously been marketed as an otic medication.

2. What I claim as my invention is Red Ear Drops, which consists of the use of Povidone-Iodine Solution, a germicidal agent which has not previously been marketed as an otic medication.
